(19) United States
(12) Patent Application Publication
(10) Pub. No.: US 2026/0207357 A1
(43) Pub. Date: Jul. 23, 2026

(54) PROSTHETIC HAND

(71) Applicant: OTTOBOCK SE & CO. KGAA, Duderstadt (DE)

(72) Inventors: David RAß, Duderstadt (DE); Reinold Philipp DURSTBERGER, Duderstadt (DE)

(21) Appl. No.: 19/140,282

(22) PCT Filed: Dec. 18, 2023
(86) PCT No.: PCT/EP2023/086457

§ 371 Date: Jun. 17, 2025

(30) Foreign Application Priority Data
Dec. 19, 2022 (DE) 10 2022 133 976.9
Aug. 28, 2023 (DE) 10 2023 123 060.3

(51) Int. Cl. A61F 2/80; A61F 2/50; A61F 2/58

(52) U.S. Cl. CPC A61F 2/80; A61F 2/588; A61F 2002/5053

(57) ABSTRACT

The invention relates to a prosthetic hand comprising an elastic prosthetic socket, having a socket wall and a proximal insertion opening to a receiving space for receiving an arm stump or a rigid socket, and also comprising a distal end portion formed distally on the prosthetic socket, the distal end portion(18) being in the form of a functional element.

The invention relates to a prosthetic hand comprising a resilient prosthesis socket which has a socket wall and a proximal introduction opening into a reception space for receiving an arm stump or a rigid socket sleeve, and comprising a distal end portion which is molded distally on the prosthesis socket.

Prosthetic hands are used to replace the shape and/or function of a hand which is not present, or no longer present. For this purpose, prosthetic hands are provided in a variety of functionalities and shapes.

Sensor-controlled prosthetic hands have a high functionality and may be constructed in technically different ways.

WO 03/017880 A1 relates to a prosthetic hand in which each individual prosthetic finger, which is supported on a chassis, respectively has a separate drive. The drive is arranged in the respective prosthetic finger. With such a prosthetic hand, it is possible to produce various gripping situations, for example fingertip gripping or lateral gripping. A disadvantage with this is the high control outlay for each individual finger, the elaborate technology with drives integrated in the fingers, and an increased susceptibility to faults because of the complex construction.

DE 405 871 B1 describes an artificial hand comprising a chassis or palm, on which rotatable fingers and a rotatable thumb are arranged. In the palm, a drive disk that can be rotated about an axis perpendicular to the volar face is attached to the fingers and the thumb by connections, in such a way that rotation of the disk in one direction causes the fingers to open and rotation in the opposite direction causes them to close. The rotation of the disk in the one direction is caused by a cord, and the restoring movement in the opposite direction is caused by a spring in the interior of the disk. Ratchet teeth, which are brought into engagement with a pawl, are applied to the circumference of the drive disk and retain the drive disk in the position that it has reached in the course of the rotation by the cord.

US 2009/0016851 A1 relates to a robotic hand comprising a base, a motor supported on the base and prosthetic fingers, which are supported on the base and articulated relative thereto. The thumb can be pivoted about two different pivot axes relative to the chassis.

Such prosthetic hands offer a good approximation to a natural hand both visually and functionally. Because of the required drives and moving parts, the fields of use of such mechanically elaborate and mobile prosthetic hands are limited. The complex design comprising a multitude of constituent parts entails high assembly outlay and furthermore necessitates a protective coating to protect the mobile components.

Comparatively simply constructed gripping elements, or so-called “hooks”, consist of two mutually displaceable hook-shaped elements that can be displaced toward one another and away from one another by a motor or by a movement of the arm or shoulder. A basic functionality is therefore provided, but there is no natural impression.

US 2004/00195638 A1 discloses a two-finger gripper in which two gripping devices can be displaced from an open position into a closed position, in which the gripping devices lie directly opposite one another. An object located between the gripping devices can therefore be held. In order to release the grip, a rotational direction reversal of the drive is induced.

So-called passive prosthetic hands, which merely imitate the appearance of a natural hand, are prosthetic hands having a very low functionality.

WO 2006/107 303 A1 discloses a prosthetic hand comprising a hand part which is shaped so that a substantially spherical ball can be held. The ball is configured so that it can be held inside the hand part and has means for fastening a tool or an object to it or in it.

The website https://www.pohlig.net/alltagshilfen describes individual functional aids for amputations or congenital malformations in the region of the upper extremity, the purpose of which is to provide a tool for carrying out frequently recurring activity. An eating aid offers the possibility of holding cutlery, while a writing aid makes it possible to control pens. For this purpose, a resilient sleeve has a receptacle for a pen or a piece of cutlery.

It is an object of the present invention to provide a lightweight prosthetic hand which is easy to use and has basic functionalities, and with which essential activities can be carried out in everyday situations.

This object is achieved with a prosthetic hand having the features of the main claim. Advantageous configurations and developments of the invention are disclosed in the dependent claims, the figures and the description.

The prosthetic hand comprising a resilient prosthesis socket which has a socket wall and a proximal introduction opening into a reception space for receiving an arm stump or a rigid socket sleeve, and comprising a distal end portion which is molded distally on the prosthesis socket, is distinguished in that the distal end portion is configured as a functional element. The distal arrangement of the end portion as a functional element, which because of the resilient configuration of the prosthesis socket can easily be fitted to the upper arm stump or lower arm stump or a corresponding reception apparatus, for example a sleeve-like socket, imparts a basic functionality in everyday situations to the hand prosthesis so that, for example, things can be clamped or held. Such a configuration is advantageous in particular for activities in so-called wet areas, in which myoelectrically controlled prostheses or prostheses with complex mechanical designs reach their limitations. With such a prosthetic hand, the lower arm stump or the replacement of a lower arm stump, or upper arm stump, can be lengthened and modified in such a way that the desired functionality is provided. Users can carry out a multitude of activities with the functional element, for example personal hygiene, cooking, for activities in wet areas, for sport or the like, without increased wear on mechanical components supported on and articulated to one another being expected. The functional element may be configured differently according to the situation. Users may have several prosthetic hands, which may be fitted as required.

In one configuration, the prosthesis socket is configured in the form of a sleeve and in such a way that it can be rolled up, so that in order to fit the prosthetic hand it is merely necessary to introduce or put the stump or the end to be received into the reception space, or into a distal end region of the reception space, and to unroll the prosthesis socket onto the lower arm stump, upper arm stump or a rigid socket sleeve. The unrolling in order to take off the prosthetic hand also offers the possibility of cleaning the prosthesis socket easily since the inner side of the reception space is thereby turned outward and is accessible.

The reception space in one configuration has a distal end region, in particular a closed distal end region, from which the functional element protrudes distally beyond the end region. The end region therefore forms a distal continuation of the prosthesis socket, and therefore also of the lower arm stump or the socket sleeve. The shape and the length of the end portion are advantageously adapted to the respective intended use and the physical circumstances of the respective user.

At least one recess, which is configured in particular as a window, is in one configuration present in the socket wall. The window is used for better securing when the inner socket is fitted to an upper or lower arm stump, since muscle tissue and fat tissue is pressed into the window because of the compression of the prosthesis socket and possibly also projects beyond the outer surface of the prosthesis socket. This prevents, or at least impedes, rotation and/or sliding of the upper or lower arm stump out of the reception space of the prosthesis socket. Furthermore, the window allows the prosthetic hand to be hung from a hook or the like when it needs to be dried after cleaning or when it is temporarily put away after replacement with another prosthetic hand for a different intended purpose.

An intermediate region, in which at least one mount for at least one tool or at least one mating piece is arranged or formed, is in one configuration present between the prosthesis socket and the distal end portion. Positioning the mount in an intermediate region between the end portion and the distal end of the prosthesis socket allows good guiding and good force transmission without impairing the functionality of the end portion. The tool or the mating piece is held securely in the mount and can be replaced according to the intended purpose. A corresponding configuration of the mount, for example with form-fit elements or clamping devices, ensures easy replaceability and secure supporting of the mating piece or tool on the prosthesis socket.

The mount in one configuration protrudes through the intermediate region so that, for example, there is accessibility of the mating piece and adjustability on the side of the back of the hand, while the tool or mating piece protruding through the intermediate region can be adjusted in its effective length, or orientation. The tool or the mating piece is therefore readily accessible for the user, for example in order to readjust it with the hand of the contralateral side. At the same time reliable support and bracing are achieved over a large distance inside the intermediate region, so that absorption of force over a large area and good force distribution take place.

The mount is in one configuration arranged or configured in such a way that a tool or mating piece held therein is positioned at a distance from the end portion or, in an alternative configuration, bears on the end portion. If the tool or mating piece is configured and positioned in the mount so that the end of the tool or mating piece does not bear on the end portion, a forked configuration is formed. An object may be hooked or placed in the free space between the noncontiguous end regions, for example in order to take household objects from a shelf, to clamp objects between them or to provide security for objects resting on the end portion. In the alternative configuration, the end region of the tool or of the mating piece bears on the end portion so that an enclosed free space is formed. The contact may be configured for stabilization of the end portion. Likewise, in a resilient configuration of the end portion and/or of the mating piece or tool, or resilient supporting in the mount, the contact may be released and an object may be placed into the free space and then resiliently held.

In one configuration, the end portion is resiliently configured, which may be advantageous when performing activities in everyday situations. In a corresponding configuration of the end portion as a functional element, it may serve directly as a kitchen utensil, for example in order to take food out of containers or put it into them, to clamp objects firmly on a tool or mating piece and/or to achieve a flexible behavior pattern, in order to prevent injuries.

The end portion consisting of a resilient material, in particular a resilient plastic or elastomer material, may in one configuration have various elements that are arranged on the end portion or inside the end portion. In order to increase stability, at least one reinforcing element may be arranged on or in the end portion. A separate reinforcing element, which is secured permanently on or in the end portion, has the advantage that the functional element and the end region can be adapted to the respective intended use in terms of stability. The material of the end portion, preferably resilient plastic, is therefore not responsible by itself for providing a sufficient geometrical stability. A local high stability of the functional element may thereby be provided, together with a protective effect by the resilient sections which surround the reinforcing element at least partially, advantageously fully. Embedding the reinforcing element or the reinforcing elements in the plastic material provides on the one hand full protection of the reinforcing element and on the other hand cushioning, which prevents damage due to hard impact of the reinforcing element on other body parts or objects. Alternatively or in addition, at least one fastening element is arranged in the end portion, for example a screw thread, a threaded insert, a sleeve with undercuts or form-fit elements, in order to be able to secure further components therein or thereon. In this way, the prosthetic hand may be modularly configured and adapted to the respective intended use. As an alternative or in addition, a shape-memory element that returns the end portion into a starting position after a deformation and activation is arranged on or inside the end portion. A spring, particularly in the form of a leaf spring, may likewise alternatively or in addition be arranged on or in the end portion, in particular embedded, in order to influence the resilient properties and particularly to provide an increased stability against deformation. In order to widen the field of use of the prosthetic hand, in one configuration a magnet is arranged on or in the end portion, either by itself or in combination with one of the aforementioned components. The at least one magnet allows force-fit securing of a magnetic or ferromagnetic component on the prosthetic hand, either in order to fix this component to the prosthetic hand or to reinforce an association with the prosthetic hand.

In one configuration, the end portion has a flat end part comprising a dorsal portion and a palmar portion. The end part is constructed substantially as a disk and has, for example, the outline of a closed hand with extended or slightly flexed fingers. The end part is therefore provided with a certain thickness, which corresponds for example to the thickness of the fingers. The end part may be configured to be straight or curved. The upper side or dorsal side is advantageously configured smoothly, while in one configuration at least one form-fit element is arranged on the palmar side, which corresponds to the inner face of the hand, and may be molded or fastened thereon. The form-fit element is used for example as an opposing bearing when objects are intended to be held or received free space located between the end portion and the mating piece. Instead of a smooth surface, elevations or depressions may be formed on the dorsal side in order to provide the upper side with a structure. Form-fit elements or projections may also be arranged on the dorsal side in order to adapt the functionality of the end portion.

A lower part, which forms a free space between it and the end part, is in one configuration arranged on the opposite side from the end part, in a similar way to the thumb side of a natural hand. The free space is then formed no longer by a mating piece, or no longer only by a mating piece, but rather by a lower part which is molded on or permanently secured by material bonding. Objects may be received inside the free space. The lower part provides security against the object falling or slipping out of the prosthetic hand. In a configuration of the prosthetic hand with the lower part, the end part forms an upper part. The mating piece or the tool may be arranged and fixed in a mount in addition to the lower part.

In order to provide a resilience adapted to the respective function, the material of the prosthesis socket and the material of the end portion have different Shore hardnesses. The material of the prosthesis socket and the material of the end portion may for example be silicone or another elastomer, in which case different Shore hardnesses may be achieved by local filling with uncrosslinked or partially crosslinked silicone having different Shore hardnesses and by combined crosslinking. If the prosthetic hand is manufactured in the scope of an additive manufacturing method, different Shore hardnesses may result from the use of different materials or different types of materials, which are applied in the respective application layer or crosslinking layer. Different Shore hardnesses may also be achieved by the use of different base materials and/or catalysts or crosslinking agents. The different hardnesses are formed in particular between the prosthesis socket and the end portion, there being a lower hardness on the prosthesis socket in the region where it is fitted to a stump than in the end portion or in the transition region between the end portion and the prosthesis socket.

In one configuration, the prosthesis socket has a palmar region and a dorsal region, the dorsal region having a higher Shore hardness than the palmar region. The palmar region is a continuation of the inner side of the hand and extends from the distal end or front end of the prosthetic hand as far as the proximal end, and the dorsal region is a continuation of the back side of the hand. In particular, configuring a harder portion on the dorsal side gives rise to a reinforced spine over the entire length of the prosthetic hand and of the prosthesis socket, which may terminate in a tongue in the region of the proximal edge of the prosthesis socket. Because of the strips of harder material on the back side of the prosthesis socket, the stump can be introduced more easily into the reception space and the prosthesis socket can be put onto the stump more easily. The harder material on the dorsal side of the prosthesis socket provides a good geometrical stability, while the comparatively softer material on the palmar side or inner side of the prosthesis socket ensures a good fit and support on the stump.

In one configuration, a coating is applied on the outer side of the prosthetic hand, particularly on subregions of the outer side of the prosthetic hand, in order to adapt the surface properties. The coating is in particular a CVD coating with which the friction of the surfaces can be reduced. The friction-reducing coating improves the haptics and, in particular, facilitates the rolling up and unrolling in the region of the prosthesis socket. In some regions, in one configuration, no friction-reducing coating is applied since, for example, an increased grip may be advantageous on the palmar side. If the prosthetic hand consists of a material that has the desired properties in this region, a coating is not applied there, while if an increased grip is desired a coating having an increased coefficient of friction or improved adhesion properties may be applied at the positions intended therefor.

In one configuration, the prosthesis socket has at least one chamber comprising a valve, for example in order to admit air or a liquid into the chamber or discharge it from the chamber. Adaptations may thereby be carried out in the region of the circumference and of the application pressure in the prosthesis socket or alternatively in the region of the end portion. A chamber without a valve may be used to receive a further element, for example a stiffening element, a balloon, a foam, a band, a magnet or the like.

The prosthesis socket and the end portion are advantageously formed integrally or connected to one another by material bonding, for example in the scope of an additive manufacturing method, a casting process, by casting plastics onto already partially crosslinked components or by adhesive bonding. An end portion molded on the prosthesis socket is intended to include all materially bonded connections between the end portion and the prosthesis socket, in particular integral primary forming, casting, adhesive bonding or welding.

According to the method for producing a prosthetic hand as described above, it is produced in one piece and a prosthetic hand is filled from at least one gate point, in particular at least one gate point on the end side, particularly a proximal gate point, against the force of gravity. The prosthesis mold forms a cavity, which is filled with the material of the prosthetic hand. The prosthesis mold is placed vertically or substantially vertically, with the distal end portion lying above the proximal end of the prosthesis socket. Provided at the lower end of the prosthesis mold, there is at least one introduction opening or at least one gate point through which the material, in particular silicone or another elastomer material, is supplied. If there are a plurality of gate points or introduction openings, these are arranged or formed in an end region at the lower end of the mold and are arranged distributed over the circumference. The gate point or the gate points is or are in particular formed in the mold in the proximal region of the prosthetic hand and allow filling against the force of gravity. The terms proximal and distal refer to the finished prosthetic hand. Besides the gate point on the end side or the gate points on the end side, further gate points may be arranged in the mold, which are spaced apart in the longitudinal extent from the gate points on the end side, in particular positioned further distally, so that material can be introduced into different planes. If only a single material hardness is desired for the prosthetic hand, the prosthesis mold is filled fully with the material. If a plurality of different zones having different material hardnesses are intended for the prosthetic hand, in one configuration a first material is initially introduced into the prosthesis mold, the first material in a crosslinked state having a higher Shore hardness than a second material in a crosslinked state. The second material is subsequently introduced onto the first material in the prosthesis mold and displaces, or transports, the first material in the distal direction of the prosthesis mold until the prosthesis mold is fully filled or until a sufficient amount of first material has emerged from an outlet opening. The first material may initially fill the cavity inside the prosthesis mold fully. By the use of different materials, which crosslink inside the prosthesis mold, a prosthetic hand in one piece having different Shore hardnesses is achieved.

The at least one gate point is advantageously located on the palmar or inner side of the prosthesis mold, so that a somewhat harder zone is formed on the outer or dorsal side of the prosthesis socket since the first material, which is harder in the crosslinked state, is pushed away from the gate point. The zone comprising the harder material advantageously extends over the entire length of the prosthetic hand, there being a gradual transition of the hardness in the palmar to dorsal direction and in the proximal to distal direction, in which case the distal end region may have a continuous uniform Shore hardness. The different hardnesses are achieved by differing amounts of the different materials and allow reproducible generation of a prosthetic hand having different hardness regions. The different materials may also be the same silicone but with differing degrees of crosslinking, so that different Shore hardnesses result in the fully crosslinked state.

Exemplary embodiments of the invention are explained in more detail below with the aid of the figures, in which:

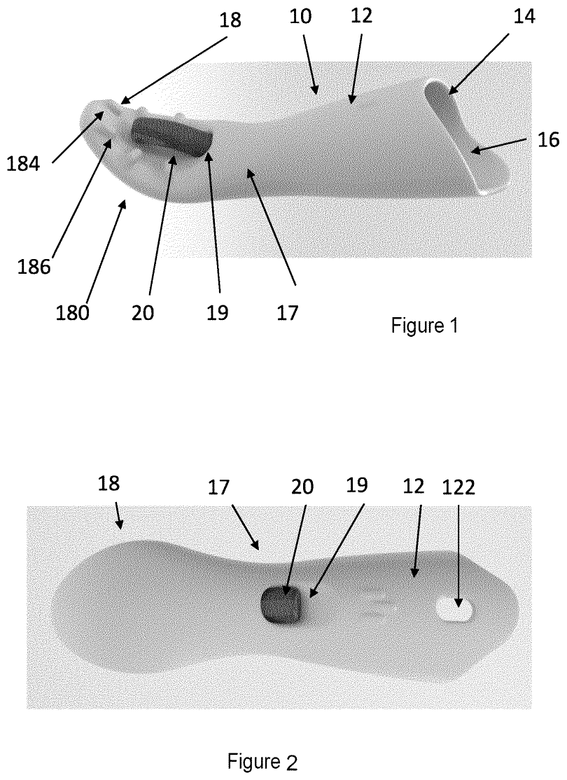

FIG. 1 shows a perspective representation of a prosthetic hand;

FIG. 2 shows a dorsal plan view of the prosthetic hand according to FIG. 1;

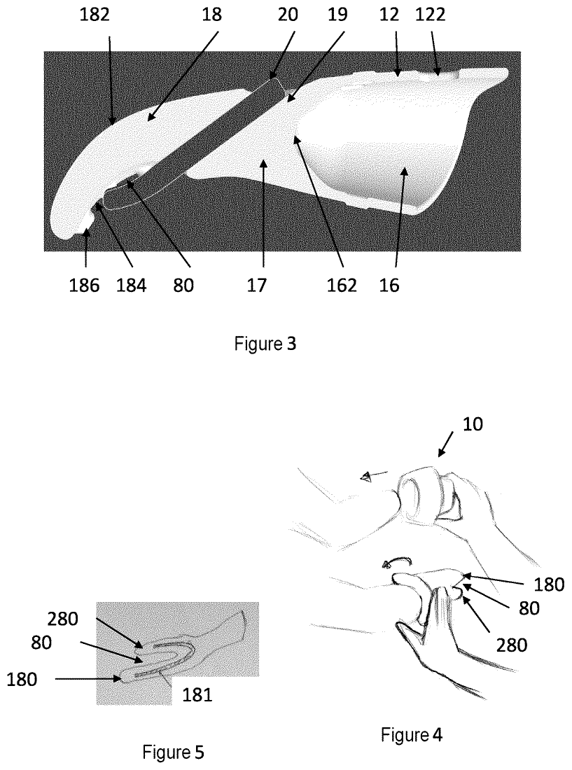

FIG. 3 shows a longitudinal sectional view of the prosthetic hand of FIG. 2;

FIG. 4 shows a variant of the prosthetic hand during fitting;

FIG. 5 shows a variant comprising a spring;

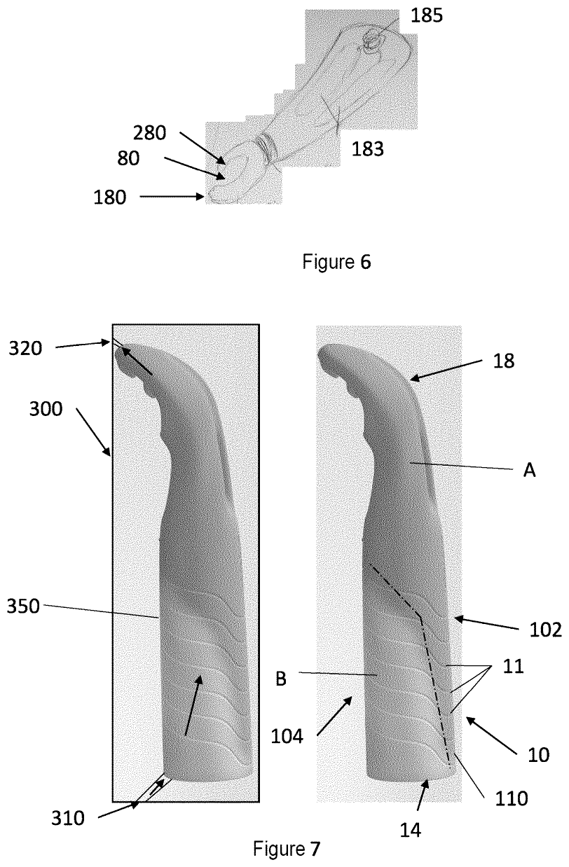

FIG. 6 shows a variant comprising chambers in the prosthesis socket;

FIG. 7 shows a schematic representation of a production method;

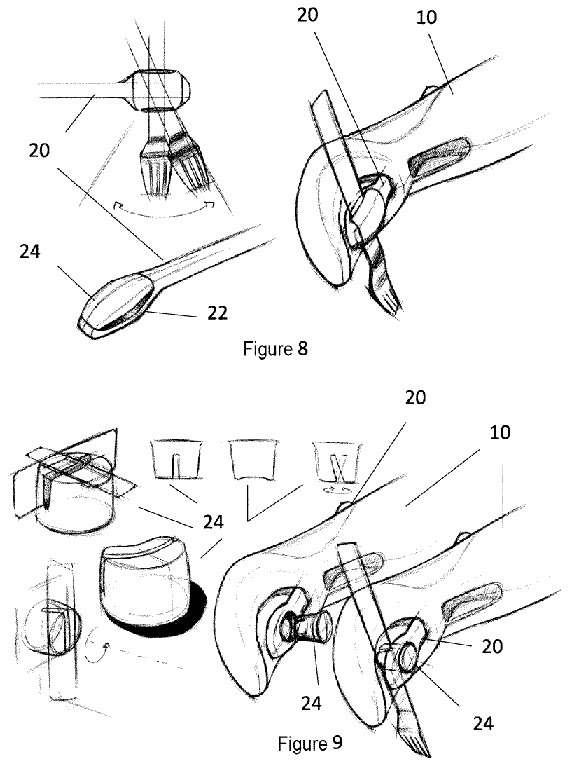

FIG. 9 shows variants of the configuration according to FIG. 8;

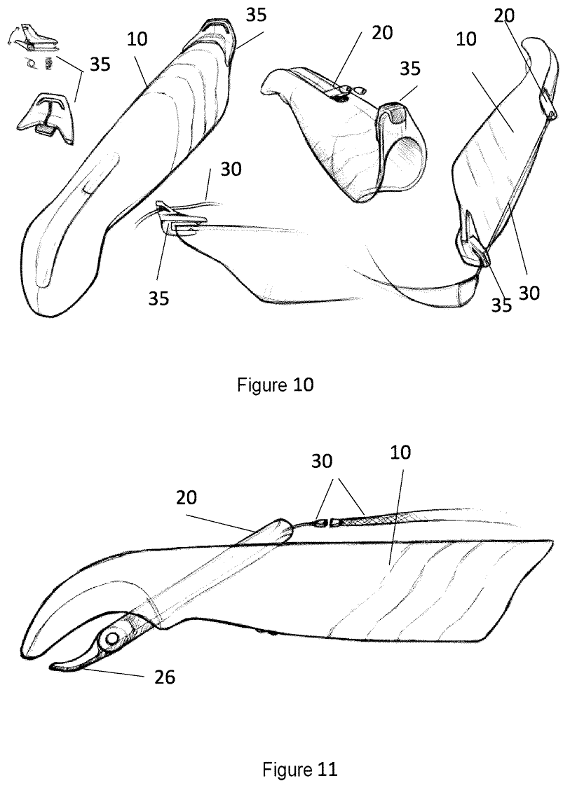

FIG. 10 shows a prosthetic hand comprising a control wire guide;

FIG. 11 shows a side view of FIG. 10;

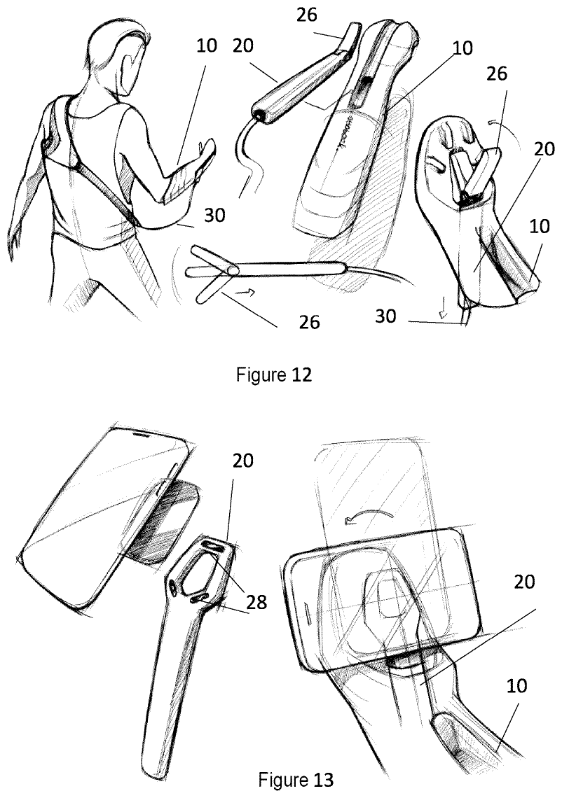

FIG. 12 shows embodiments of a prosthetic hand comprising an actuation device;

FIG. 13 shows a variant of the prosthetic hand comprising a mount;

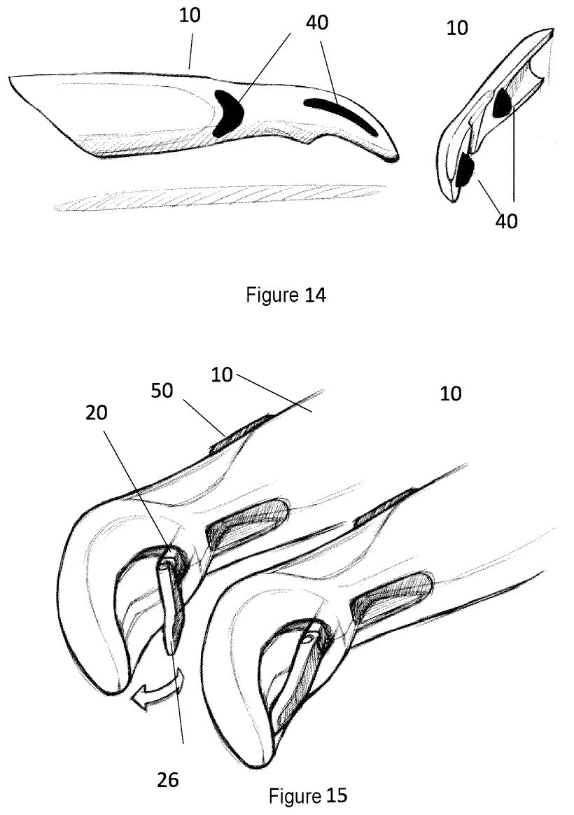

FIG. 14 shows sectional representations of a prosthetic hand comprising inlays;

FIG. 15 shows a prosthetic hand comprising a mechatronic tool;

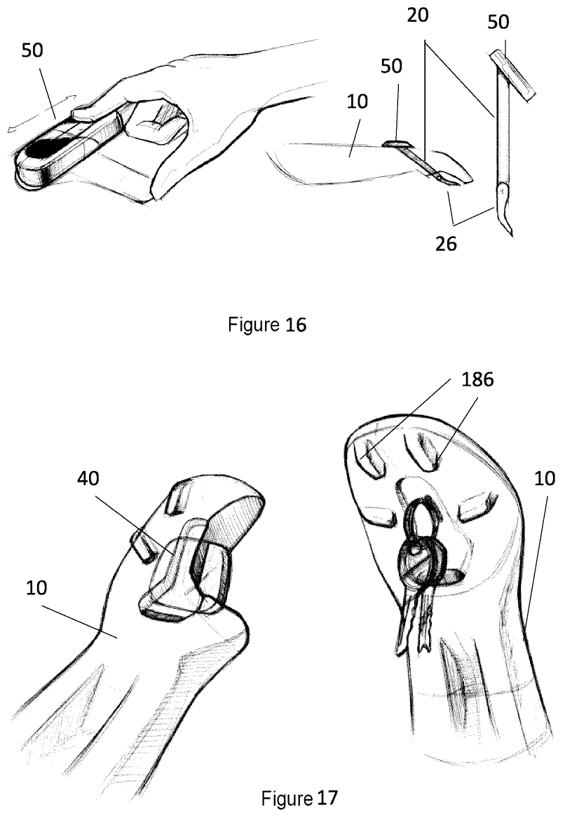

FIG. 16 shows a sectional representation through a prosthetic hand comprising a mechatronic tool and an operating element;

FIG. 17 shows a prosthetic hand comprising a magnetic inlay;

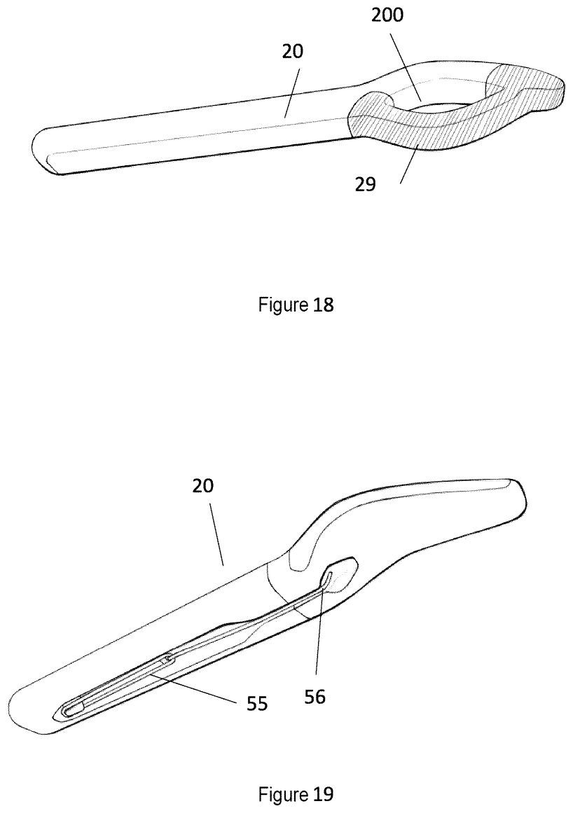

FIG. 18 shows mating piece comprising springs of the component;

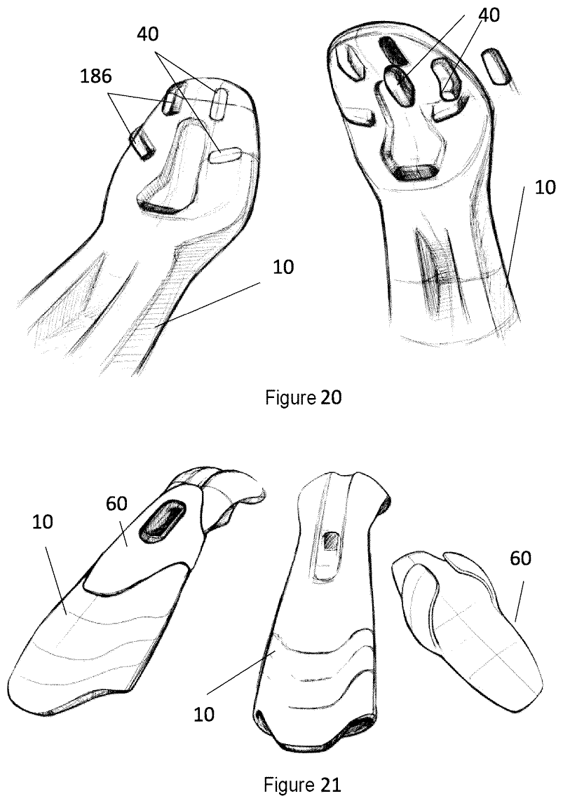

FIG. 19 shows a prosthetic hand comprising a spring element;

FIG. 20 shows a prosthetic hand comprising palmar inserts;

FIG. 21 shows a prosthetic hand comprising a splint;

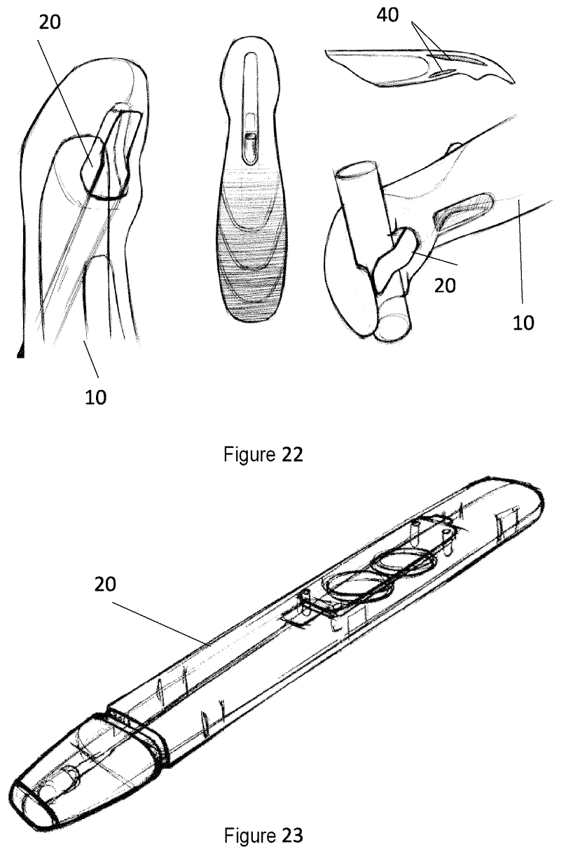

FIG. 22 shows a prosthetic hand consisting of a soft base material with inserts consisting of a harder material;

FIG. 23 shows a mating piece in the form of a flashlight;

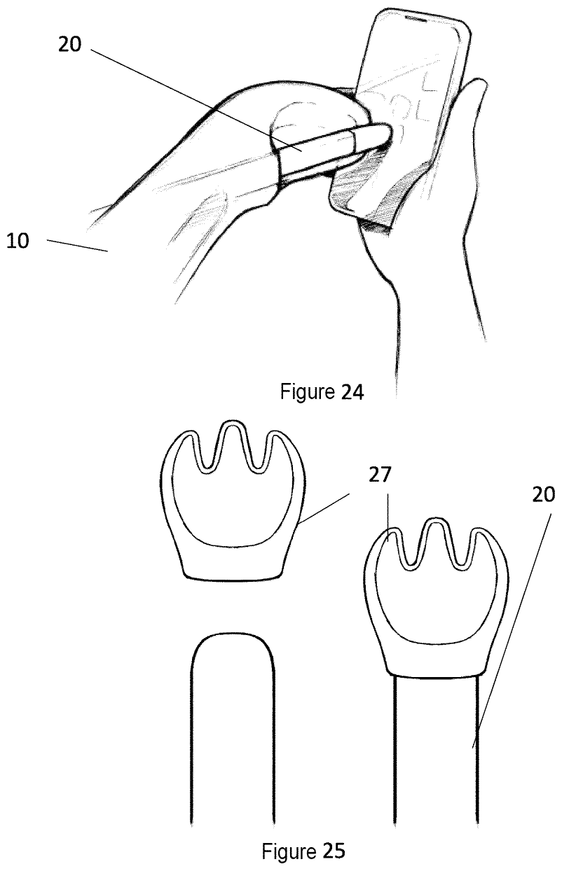

FIG. 24 shows a prosthetic hand comprising a touch tool;

FIG. 25 shows a variant of a mating piece; and

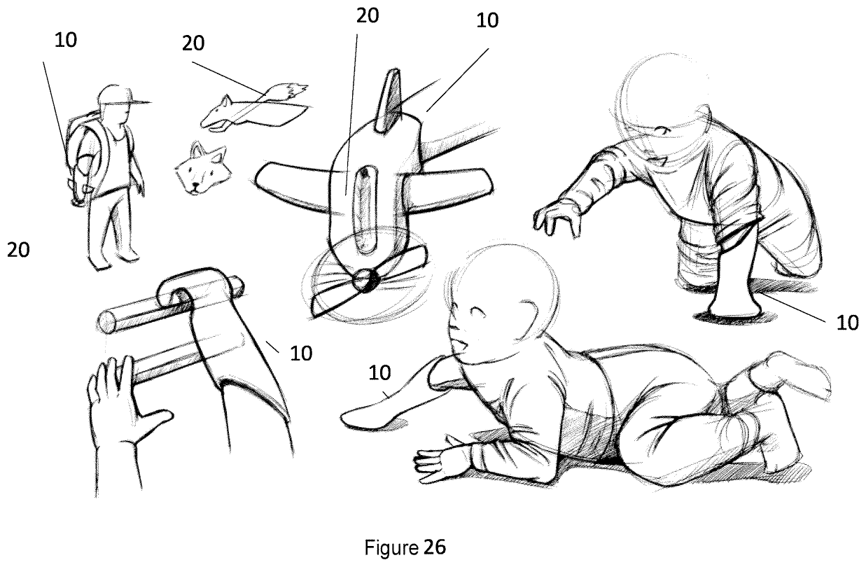

FIG. 26 shows variants of a prosthetic hand for infants.

FIG. 1 represents a perspective view obliquely from below of a prosthetic hand in an integral configuration, which has a sleeve-like prosthesis socket 10 comprising a socket wall 12 and a proximal introduction opening 14. The socket wall 12 forms a reception space 16 into which an arm stump can be introduced, in particular directly or alternatively after fitting a covering, a liner or a textile covering. Alternatively, the prosthesis socket 10 is used to receive a socket body in the form of a sleeve, for example a cuff or a clasp, which may be configured rigidly or stiffly, such as a prosthesis socket of a myoelectrically controlled prostheses without the gripping appliance mounted, and can be placed around a lower arm stump. The socket sleeve, which is not represented, is used for example for stabilizing the tissue, for shaping or for lengthening a lower arm stump. The reception space 16 is configured to be substantially closed and has a closed distal end region, which may be seen more clearly in FIG. 3. The reception space 16 is followed by an intermediate region 17, which is solidly configured and opens into an end portion 18, and is positioned distally with respect to the prosthesis socket 10 and the reception space 16 and distally with respect to the intermediate region 17. The end portion 18 forms the distal extremity of the prosthetic hand and is configured substantially as a disk in the embodiment represented, and forms an end part 180 having a contour that corresponds to a hand with closed, slightly bent fingers. The end portion 18 forms a spoon-shaped functional element. A thumb element is not formed from the material of the end portion 18. On the end part 180, the end portion 18 has a palmar portion 184 which corresponds to the inner side of the hand. The palmar portion 184 on the end part 180 has projecting form-fit elements 186, in the exemplary embodiment represented six form-fit elements 186, which are configured as blocks or bars and project beyond the otherwise substantially smooth surface of the palmar portion 184. The form-fit elements 186 in the exemplary embodiment represented are directed outward and arranged on the circumference of the end part 180. Centrally between the form-fit elements 186, there is a mating piece 20 which has been produced from a different material than the prosthetic hand 10, in particular from a rigid geometrically stable material. The mating piece 20 is inserted into, and held in, a mount 19 which is configured as a channel through the intermediate region 17. The mating piece 20 may be positioned and fixed in any desired way inside the mount 19. For this purpose, the mating piece 20 is guided through the channel from the dorsal side, as may be seen in FIG. 2, and is held in the respectively desired position because of resilient holding forces, friction forces or by form-fit elements. In the exemplary embodiment represented, the end region of the mating piece 20 bears on the surface of the palmar portion 184 between the form-fit elements 186 so that a free space, which may be used to receive objects, is formed between the mating piece 20 and the palmar portion 184 owing to the curved shape of the end part 180. The objects are for example introduced through the intermediate space between two form-fit elements 186 into the free space between the mating piece 20 and the palmar portion 184, and held in this region owing to the resilient configuration. Because of the mutually opposite arrangement of the form-fit elements 186, it is possible for a rod, a screwdriver, a shaver or a spoon, for example, to be held in the respectively desired and adjusted orientation, for example perpendicularly to the longitudinal extent of the mating piece 20, at a 45° angle thereto or substantially parallel thereto as a continuation of the mating piece.

FIG. 2 represents a plan view from a dorsal view, in which the accessibility of the separate rigid mating piece 20 from the dorsal side of the prosthetic hand may be seen. The mount 19 comprising the channel extends through the intermediate region 17 and protrudes through it. The intermediate region 17 corresponds substantially to the region of the wrist of a natural hand. Inside the intermediate region 17 and the end part 180, in particular, reinforcing elements may be arranged in order to limit the resilience, or increase the restoring force, and achieve an increased geometrical stability of the end part 180 and of the intermediate region 17. In the proximal end region of the prosthesis socket 10, a recess 122 in the form of a window is arranged in the socket wall 12, which is helpful when putting on the prosthesis socket 12 as well as when taking it off, can be used for hanging up the prosthetic hand when it has been taken off, and assists with fixing the prosthesis socket 10 to the lower arm in the fitted state.

FIG. 3 shows a longitudinal sectional representation through a prosthetic hand according to the embodiment of FIGS. 1 and 2. Besides the integral configuration consisting of a silicone or a copolymer, the reception space 16 with its distal end region 162 may be seen in the sectional representation. The reception space 16 is therefore configured as a closed sleeve comprising a closed end region 162 and an introduction opening. The socket wall 12 is provided in the proximal end region with an opening 122 in the form of a window, and compressed muscle tissue or soft tissue can penetrate through the window or the opening 122, or protrude into the recess 122, and possibly project beyond the outer side of the socket wall 12. In the sectional representation according to FIG. 3, the mount 19 in the form of a channel extending through the intermediate region 17 may be seen. The rod-shaped mating piece 20, which is bent at the end region, is inserted inside the mount 19, and its front end bears on the palmar portion 182 so that a free space 80, which may be used to receive objects, is formed between the surface on the palmar side of the end part 180 and the mating piece 20. Because of the resilient configuration of the end portion 18, the end portion 18 can spring back after a deformation and clamp the object between it and the rigid mating piece 20. The mating piece 20 is arranged replaceably inside the mount 19 and fixed therein. The end portion 18 is configured as a functional element even without a mating piece 20 owing to the bent, spoon-like shape with the smooth dorsal portion 182. The form-fit elements 186 are used for easier fixing and firm clamping of objects.

FIG. 4 represents the fitting of the prosthetic hand directly to a lower arm stump. The prosthesis socket 10 is rolled up, or folded, from its proximal end in the direction of the end portion 18 so that the distal end region 162 is readily accessible. The distal stump end is inserted into the closed distal end region 162, as is shown in the upper representation of FIG. 4. The forward-folded or rolled-up prosthesis socket 10 is subsequently unrolled onto the lower arm stump, which is indicated in the lower representation of FIG. 4. The prosthesis socket 10 bears on the lower arm stump over the entire circumference of the lower arm stump, with the exception of a possible recess 122, and secures the prosthetic hand with the end region 18 to the lower arm stump. The embodiment according to FIG. 4 shows the end region comprising an end part 180 and an opposite lower part 280, which are formed in one piece from the same material. Formed between the end part 180 and the lower part 280, there is a free space 80 which is used to receive objects or may serve other purposes. The distal ends of the end part 180 and of the lower part 280 may, for example, be employed to wash hair or to come in contact with other surfaces, and may be configured so that the prosthetic hand can be stood on them when it has been taken off.

FIG. 5 shows a schematic representation of the prosthetic hand comprising an end part 180 molded on and a lower part 280 molded on. An elastic material, a spring or a shape-memory material 181, which extends into the distal regions of the end part 180 and of the lower part 280, is cast inside the end portion 18. The spring 181 is used at the same time as a reinforcing element and allows secure holding of objects clamped between the lower part 280 and the end part 180.

FIG. 6 represents a prosthetic hand comprising the prosthesis socket 10, inside which two chambers 183 that can be filled with air through a valve 185, with which a pump may be associated, are formed. Conversely, air is discharged through the valve 185 so that the chambers 183 can be emptied and the prosthesis socket 10 can easily be fitted and taken off. When the chambers 183 are filled with air, the application pressure on the lower arm socket increases and the prosthesis socket is pressed tightly onto the lower arm stump in order to obtain a secure fit.

In the case of dysmelia patients or patients with scar tissue, there may be excess structures on parts of the stump, for example keloids, calluses or incompletely formed fingers. These structures are often present on the distal region of the stump and may be accommodated by gaps or recesses in the socket wall 12 or socket receptacle, particularly in the distal end region 162 of the reception space 16.

FIG. 7 schematically represents the production method of a prosthetic hand comprising a prosthesis socket 10. The left representation shows a prosthesis mold 300 comprising a cavity 350, which corresponds to the outer shape of the prosthetic hand with the prosthesis socket 10. In order to form the reception space 16 (not represented) for receiving the arm stump through the proximal introduction opening 14, a corresponding insert is arranged or formed inside the cavity 350. A first material A is initially injected or introduced into the cavity 350 through a gate point 310. A plurality of gate points 310 may be arranged distributed over the circumference at the lower end of the mold and allow the supply of material into the mold. Different material may be introduced through each gate point. The prosthesis mold 300 stands substantially vertically, so that filling takes place upward from below against the force of gravity, which is indicated by the arrows marked in the prosthesis mold 300. The first material A, having a comparatively high Shore hardness in the fully crosslinked state, is introduced into the prosthesis mold 300 until the cavity 53 is fully filled, or until material emerges from an upper vent opening or exit opening 320. A defined amount of a second material B is subsequently introduced into the prosthesis mold 300 through the gate point 310, the gate point 310 being positioned in the region of the proximal edge of the prosthesis socket on the palmar side, that is to say the inner side of the prosthesis socket. The second material B then gradually displaces the first material A, the material A in the vicinity of the gate point 310 being displaced first by the material B, which is softer in the crosslinked state, because of the position of the gate point 310. The set amount of the second material B introduced dictates the degree of displacement of the harder material A. As an alternative to filling the mold 300 fully with the first material A, the mold 300 may also be filled only partially with the first material 300 and subsequently filled up with the softer material B.

After the complete and final filling of the prosthesis mold 300 with the material having different hardnesses or with the different materials having different hardnesses, the filling process is ended and the material is crosslinked, or the materials are crosslinked. This creates a prosthetic hand in one piece, which in the fully crosslinked state has different regions with different hardnesses. In particular, in the region of the prosthesis socket 10 on the back side of the hand in a dorsal region 102, the prosthesis socket 10 and the prosthetic hand have a higher Shore hardness than in a palmar region 104. In the right representation of FIG. 7, in which the fully formed prosthetic hand is represented without the mold 300, this is indicated by the dashed line which represents a transition region in which the softer material B mixes with the harder material A. A spine or a rib of the harder material A is formed on the dorsal side 102, while a soft resilient region is formed from the material B in the palmar region on the lower arm. The end portion 18 consists entirely of the harder material A and is preferably configured solidly. A greater stability is attainable by the higher Shore hardness in the distal end region 18 in order to achieve a greater clamping force when a mating piece 20, for example a thumb part, is inserted.

At the proximal end of the prosthesis socket 10, a flap 110 may be formed in the dorsal region, on which the user can grip the stable flap 110 consisting of the harder material with the untreated hand. The softer material region in the palmar region 104 makes it easier to fit and take off the prosthesis, and offers more possibilities of adapting to different stump shapes. Material weakenings 11 or curved lines may be seen on the outer side of the prosthesis socket 10, which allow shortening of the prosthesis socket 10 along the material weakenings 11. The shape of the material weakenings 11 is selected so that the flap 110 resulting after the shortening forms the proximal end of the prosthesis socket 10 and the socket edge extends in a curved shape with a length reduction in the dorsal region 104. The represented distribution of the material, or of the different hardnesses of the materials A and B, is an exemplary distribution and other profiles may also be envisioned. Likewise, more than two different materials having different Shore hardnesses in the crosslinked state are possible.

FIG. 8 presents representations of a variant of a prosthetic hand comprising a prosthesis socket 10 and a mating piece 20, which is configured as a mount for a cutlery element. For this purpose, the mating piece 20 has a shaft which is inserted and received in the prosthesis socket 10. The shaft of the mating piece 20 in the exemplary embodiment represented protrudes through the prosthesis socket 10 and has at its distal end a receptacle 22 into which a tool, for example a fork, a spoon or a knife, can be inserted. The receptacle 22 is formed by a free space between the mating piece 20 and a holder 24. The holder 24 clamps, for example, the tool or the piece of cutlery. In the upper left representation, it may be seen that the piece of cutlery can be held and fixed in different alignments or orientations inside the receptacle 22. This may, for example, be done by the piece of cutlery being clamped between the holder 24 and the mating piece 20. There may likewise be additional safety or holding devices, for example magnet elements or form-fit devices. Alternatively, the piece of cutlery may be fixed inside or on a mount 24, which may then in turn be displaced relative to the mating piece 20, for example via latching recesses into which a holder 24 that is correspondingly spring-loaded, or prestressed by another safety device, can be latched.

FIG. 9 shows a variant of the holder 24. The holder 24 in the right representation of the prosthetic hand is likewise inserted in the mating piece 20. At its distal end, the mating piece 20 has a recess or bore into which the conically configured holder 24 is introduced. There are one or more slots in the holder 24, into one of which the piece of cutlery is inserted, the holder 24 already being located in the recess or bore. If the holder 24 is introduced further into the recess or bore inside the mating piece 20, the slot is narrowed owing to the conical configuration of the holder 24 and the piece of cutlery, in the exemplary embodiment represented a fork, is clamped. The left representations of FIG. 9 show different configurations of the holder 24. Because of a frustoconical configuration of at least a part of the holder 24, the latter is freely rotatable inside the mating piece 20 and is fixed in the respectively desired position by pressing into the recess of the mating piece 20. The rotatability about the longitudinal axis of the conic frustum is indicated by the arrows. The slots inside the holder 24 may be configured at differing angles relative to the end surfaces in order to allow different orientations of the tool or piece of cutlery.

FIG. 10 shows a prosthetic hand comprising a prosthesis socket 10 and a mating piece 20 arranged thereon, the mating piece 20 having a mobile component 26 at its distal end, which can be actuated via a control wire 30. The mobile component 26 is represented in a side view in FIG. 11 and is configured as a tip that can be displaced about a pivot axis. The mobile component 26 can therefore be displaced toward or moved away from the distal end portion 18, or the end part 180. The mobile component 26 may be spring-loaded so that the mobile component 26 returns into the starting position after a tensile force on the control wire 30 is released. A control wire guide 35 is arranged at the proximal end of the prosthesis socket 10; in the exemplary embodiment represented, the control wire guide 35 is plugged onto the proximal edge of the prosthesis socket 10. In the upper left representation of FIG. 10, it may be seen that the control wire guide 35 has a spring-loaded mechanism so that a lower flap of the control wire guide 35 is opened, the control wire guide 35 is pushed onto the prosthesis socket 10 and subsequently, after the actuation force is released, the control wire guide 35 is held by clamping. Alternatively, the lower flap is arranged or formed on the lower side of the control wire guide 35 by a film hinge or by another resilient component or a resilient bearing, and can be fitted onto the prosthesis socket 10 for firm clamping.

At least one slot, through which the control wire 30 can be guided as far as the mating piece 20, is formed inside the control wire guide 30. The course of the control wire 30 along the longitudinal extent of the prosthesis socket 10 is defined by the control wire guide 35. The control wire 30 may be configured in multiple parts, the control wire 30 in the right representation of FIG. 10 being guided through the control wire guide 35 in the distal to proximal direction and having a belt at its proximal end, which for example is connected to a contralateral shoulder or another body part, so that the control wire 30 is tensioned by a corresponding displacement of the prosthesis socket 10 away from the body and conversely relaxed by a reverse movement. As an alternative to a control wire 30, it may also be formed by a belt, cable or the like, all force transmission means that transmit tensile forces being envisioned as a control wire.

FIG. 11 shows a multi-part configuration of the control wire 30. The distal part of the control wire 30 is guided inside the mating piece 20 to the mobile component 26 via a connecting element, and the proximal component of the control wire 30 may be fastened thereto by a connecting element, for example a magnetic coupling, a clip connection, a hook, a screw connection or a combination of several of these connection principles, or by other connecting elements.

FIG. 12 schematically represents the use of a prosthetic hand comprising a driven mobile component 26 on the mating piece 20. At its distal end, the mating piece 20 has a mobile component 26 which is displaced relative to the mating piece 20 by the control wire 30. In the central representation, the mating piece 20 is not yet introduced into the prosthesis socket 10, and the left representation shows that the control wire 30 is secured to the shoulder on the contralateral side, which is not treated with the prosthetic hand, and can be tensioned or relaxed by a corresponding relative displacement. Depending on the tension, as represented in the lower representation of FIG. 12, the mobile component 26, which may also be held pretensioned in a starting position, is displaced against the spring force so that an opening or closing movement is obtained. Such a movement may be seen in the right representation of FIG. 12. If the control wire 30 is pulled in the arrow direction, the mobile component 26 is displaced in the direction of the end portion so that a closing movement is initiated and, for example, an object can be held. If the tension is reduced, the mobile component 26 correspondingly opens. Alternatively, a movement reversal will take place, in which case the mobile component 26 is held in the closed position in the event of a relaxed control wire 30 and is opened by a pull on the control wire 30.

FIG. 13 shows a further variant of the mating piece, in which a mount that in the exemplary embodiment represented makes it possible to receive a cell phone is formed at the distal end of the mating piece 20. For example, magnets 28 are arranged at the distal end of the mating piece 20 in order to hold the cell phone, or a mount of the cell phone, on the mating piece 20 with a force fit. There may also be a recess inside the mating piece 20, through which a projection or a journal is guided so that the cell phone, or another apparatus, is supported rotatably on the mating piece 20. The rotatability is shown in the right representation of FIG. 13.

FIG. 14 shows a prosthetic hand comprising the prosthesis socket 10 in cross-sectional representations. Besides the reception space and the distal end portion, inserts 40 may be seen in FIG. 14. The inserts 40 have an increased stiffness in relation to the other material of the prosthesis socket 10, or the end portion. The proximal insert 40 is configured in the shape of a bowl or cup, and has a contour corresponding to the reception space 16. An increased stability in the reception space for receiving the lower arm stump can be ensured by the insert 40. The insert 40 in the end portion increases the stiffness inside the distal end portion and acts as an effective counter-bearing for the mating piece, which is not inserted inside the prosthesis socket in FIG. 14. The inserts 40 may be produced from different materials, and the stiffnesses of the individual inserts may differ from one another so that an adapted stiffness can be adjusted in the respective regions of the prosthetic hand, or of the prosthesis socket.

FIG. 15 represents a variant of the mating piece 20 inside the prosthesis socket 10. The mating piece 20 again has a mobile component 26 at the distal end, which in the exemplary embodiment represented is configured as a mechatronic component. Arranged at the proximal end of the mating piece 20, there is an actuation element 50 via which the mobile component 26 can be displaced by a drive, which is arranged either in the prosthesis socket 10 or in the mating piece 20. The displaceability is represented by the double arrow, with the mobile component 26 being represented in an open position in the left representation. After activation of the motor drive via the actuation element 50, it can be displaced into the position according to the right representation of FIG. 15.

FIG. 16 represents the actuation element 50 in combination with the mating piece 20 in more detail. The left representation of FIG. 16 represents the actuation element 50 as a touch field or display, in which opening or closing of the mobile component 26 is caused by a swiping movement in one direction or the other. The central representation of FIG. 16 shows a sectional representation of the prosthetic hand with the actuation element 50 on the upper side or dorsal side of the prosthesis socket. The right representation shows the mating piece 20 with the actuation element 50 and the mobile component 26. Instead of a touch-sensitive operating field, the actuation element 50 may also have buttons, knobs or sliders in order to bring about a displacement of the mobile component 26 relative to the other components of the mating piece 20 and the prosthetic hand. If both the necessary control software and memories, drives and energy stores are arranged inside the mating piece 20, the latter may be manufactured separately as a modular component and inserted into the corresponding recess inside the prosthesis socket 10.

FIG. 17 shows different views of a variant of a prosthetic hand comprising a magnetic insert 40 in the palmar region of the end portion, so that magnetic or magnetizable elements, for example a bunch of keys, can be held on the inner side of the end portion. A mating piece is not arranged in the prosthesis socket 10 in FIG. 17, although one may be used in addition to the insert 40 in the prosthesis socket. The form-fit elements in the exemplary embodiment represented are arranged around the magnetic insert 40 in order to achieve additional security of the objects to be received.

In FIG. 18, the mating piece 20 is formed from different materials. A first material, which forms the proximal shaft and a curvature, is comparatively rigid, while a second material forms a spring component 29. The spring component 29 is shaped so that it forms a locally soft tip in the distal region of the mating piece and, with an opposite region of the mating piece 20 consisting of the rigid material, encloses a free space 200. The free space 200 in the exemplary embodiment represented is configured to be round, oval or in the shape of a prism. The spring component, which may for example consist of a substantially softer and flexible material such as silicone, makes it possible to clamp and hold objects between the mating piece 20 and the end portion 18 (not represented) of the prosthetic hand. The mating piece 20 with the spring component 29 and the free space 200 may be produced in an additive production method by using a plurality of different materials. Alternatively, the spring component 29 is fastened to the mating piece 20 afterward.

In FIG. 19, a mating piece 20 is produced from a plurality of materials, the distal end of the mating piece 20 being fastened replaceably to a proximal shaft. The distal end piece of the mating piece 20 is, for example, fastened to the shaft by a spring 25 on or inside the shaft. The spring may be coupled to a hook 56 so that either the distal end piece of the mating piece 20 is held on the shaft or, via the hook which is supported elastically inside the mating piece 20, a further component may be fastened to the mating piece 20 or held thereon.

In FIG. 20, different inserts 40 are arranged in the palmar region of the end portion in addition to the form-fit elements 186. The inserts 40 may either end flush with the inner face of the end portion or project beyond the inner face, so that there are additional form-fit elements or resistances as well as mounts on the inner side, or palmar face, of the end portion. The inserts 40 are arranged replaceably inside the end portion.

FIG. 21 shows a variant in which a splint 60 is fitted in the area of the intermediate region. The splint 60 stabilizes the intermediate region so that the latter provides an increased stability in relation to mechanical deformation. The splint 60 may extend over a large region of the longitudinal extent of the prosthesis socket and the intermediate region, into the end portion, and have a recess for receiving the mating piece 20 and guiding it through. By such a splint 60, which is represented separately from the prosthesis socket 10 in the right representation of FIG. 21, a high mechanical stability may be provided on a case by case basis. The splint 60 can easily be fitted onto the prosthesis socket 10 and taken off, so that the mechanical properties of the prosthetic hand can be modified easily and rapidly. The splint 60 may be configured elastically in the circumferential direction and have lateral clasps, so that the open cross section can be bent apart easily. The splint 60 may be formed from a fiber-reinforced plastic which is sufficiently lightweight and at the same time stable in order to ensure on the one hand elastic fitting and on the other hand sufficient stiffness for the prosthetic hand.

FIG. 22 represents a further variant, in which an end portion consisting of a very soft material, in particular a soft silicone, lies opposite a comparatively rigid mating piece 20, inserts 40 being embedded in the end portion 18 in order to achieve a sufficient stability. The soft configuration of the material around the inserts 40 can ensure secure gripping or reception of objects in the free space between the mating piece 20 and the end portion 18.

FIG. 23 represents a variant of the mating piece 20 in the form of an electronic component, in the example represented in the form of a flashlight. The mating piece 20 comprising suitable batteries and actuation elements may be inserted easily into the prosthetic hand and extends the possible field of use by lighting means arranged in the distal region.

In FIG. 24, a touch-tool instead of a lamp or a mechatronic element is arranged as a mating piece 20. A touch-sensitive element at the distal region of the mating piece 20 makes it possible to operate smartphones, tablets, wearables and similar touch-sensitive devices, so that operation of electronic devices or interfaces comprising touch-sensitive displays is facilitated.

FIG. 25 shows a variant of the mating piece 20 in a multi-part configuration. The mating piece 20 has a distal attachment 27, which is arranged replaceably at the end of the mating piece 20. In the exemplary embodiment represented, the attachment is configured as a fork, although it may alternatively also be configured as a spoon or another tool element which can be plugged onto the mating piece 20 as required.

FIG. 26 represents different embodiments of a hand prosthesis for children. The prosthesis socket 10 in each case has an end portion that can be adapted to the different requirements and developmental stages of an infant. Besides flattened and rounded end portions, such as are shown in the right two representations, the end portion may also be configured to be curved or clawlike as is represented in the bottom left figure, for example in order to be able to clamp sticks or other objects in the resilient intermediate space. The mating piece 20 may in one embodiment also be configured as a toy, and the end portion may optionally be configured as an image of an animal, of a vehicle or of a fantasy figure in order to increase acceptance of the prosthesis for the person using it. The entire prosthesis may have the form of an animal, of a plant or of an object differing from the shape of a natural hand.

### List of Reference Signs

10—prosthesis socket
12—socket wall
14—proximal introduction opening
16—reception space
17—intermediate region
18—end portion
19—mount
20—mating piece
22—receptacle
24—holder
26—mobile component
27—attachment
28—magnet
29—spring component
30—control wire
35—control wire guide
40—insert
50—actuation element
55—spring
56—hook
60—splint
80—free space
102—dorsal region
104—palmar region
110—flap
122—recess
162—distal end region
180—end part
181—shape-memory material
182—dorsal portion
183—chamber
184—palmar portion
186—form-fit element
200—free space
280—lower part
300—mold
310—gate point
320—exit opening
350—cavity

1. A prosthetic hand comprising a resilient prosthesis socket which has a socket wall and a proximal introduction opening into a reception space for receiving an arm stump or a rigid socket sleeve, and comprising a distal end portion which is molded distally on the prosthesis socket, characterized in that the distal end portion is configured as a functional element.

2. The prosthetic hand as claimed in claim 1, characterized in that the prosthesis socket is configured in the form of a sleeve and in such a way that it can be rolled up.

3. The prosthetic hand as claimed in claim 1, characterized in that the reception space has a distal end region and the functional element protrudes distally beyond the end region.

4. The prosthetic hand as claimed in claim 1, characterized in that at least one recess is formed in the socket wall.

5. The prosthetic hand as claimed in claim 1, characterized in that at least one mount for at least one tool or at least one mating piece is arranged or formed in an intermediate region between the prosthesis socket and the distal end portion.

6. The prosthetic hand as claimed in claim 5, characterized in that the mount protrudes through the intermediate region.

7. The prosthetic hand as claimed in claim 5, characterized in that the mount is arranged or configured in such a way that a held tool or the mating piece is positioned at a distance from the end portion or bears on the end portion.

8. The prosthetic hand as claimed in claim 1, characterized in that the end portion is resiliently configured.

9. The prosthetic hand as claimed in claim 1, characterized in that at least one reinforcing element, at least one fastening element, a shape-memory element, a spring and/or at least one magnet is/are arranged in the end portion.

10. The prosthetic hand as claimed in claim 1, characterized in that the end portion has a flat end part comprising a dorsal portion and a palmar portion.

11. The prosthetic hand as claimed in claim 10, characterized in that at least one form-fit element, which projects in the palmar direction, is molded or fastened on the palmar portion.

12. The prosthetic hand as claimed in claim 10, characterized in that the end portion has a lower part lying opposite the end part, between which and the end part a free space is formed.

13. The prosthetic hand as claimed in claim 1, characterized in that the material of the prosthesis socket and the material of the end portion have different Shore hardnesses, in particular the Shore hardness of the end portion or of a transition region being higher than that of the prosthesis socket.

14. The prosthetic hand as claimed in claim 1, characterized in that the prosthesis socket has a palmar region and a dorsal region, and the dorsal region has a higher Shore hardness than the palmar region.

15. The prosthetic hand as claimed in claim 1, characterized in that a coating is applied on the outer side of the prosthetic hand.

16. The prosthetic hand as claimed in claim 1, characterized in that at least one chamber comprising a valve or for receiving a stiffening element is formed or arranged in the prosthesis socket.

17. The prosthetic hand as claimed in claim 1, characterized in that the prosthesis socket and the end portion are formed integrally.

18. A method for producing a prosthetic hand as claimed in claim 1, characterized in that it is produced in one piece and a prosthesis mold is filled from a gate point against the force of gravity.

19. The method as claimed in claim 18, characterized in that the prosthesis mold is initially filled with a first material (A), which has a higher Shore hardness in a crosslinked state than a second material (B) in the crosslinked state, and in that the second material (B) is subsequently introduced onto the first material (A) and displaces the first material (A) in the distal direction of the prosthesis mold.
